(19) United States
(12) Patent Application Publication
(10) Pub. No.: US 2007/0074722 A1
(43) Pub. Date: Apr. 5, 2007

(54) Medicament delivery control, monitoring, and reporting system and method

(75) Inventors: Marc Giroux, Lynnwood, WA (US); William A. DeGroodt, Mill Creek, WA (US); Arthur L. Rizer, Seattle, WA (US)

Correspondence Address: DAVIS WRIGHT TREMAINE, LLP, 2600 CENTURY SQUARE, 1501 FOURTH AVENUE, SEATTLE, WA, 98101-1688, US

(73) Assignee: Kurve Technology, Inc., Bothell, WA (US)

(21) Appl. No.: 11/525,308

(22) Filed: Sep. 21, 2006

Related U.S. Application Data
(60) Provisional application No. 60/719,432, filed on Sep. 21, 2005.
(60) Provisional application No. 60/736,802, filed on Nov. 15, 2005.

(51) Int. Cl. A61M 15/00

(52) U.S. Cl. 128/203.15

(57) ABSTRACT

Particular aspects provide novel intelligent delivery devices and methods for administration of therapeutic agents, solutions, nutrients, etc. The intelligent delivery devices have substantial utility for validating medicament use, optimizing medicament delivery, storage and communication of medicament delivery-related data, etc., based on recognition and processing of identifying information associated with the Medicament and/or packaging thereof. The systems and methods are broadly applicable to medicament delivery applications and many devices. Particular aspects provide an interface between a medicament cartridge and an electronic atomizer/nebulizer used for optimized medicament delivery. An intelligent ID tag (e.g., RFID tag, magnetic strip, bar code, etc.) is associated with a medicament or its container and is recognizable by, or the information thereof can be input into an electronic sensing means of the atomizer/nebulizer, whereby validation, and optimal delivery of the agent is afforded according to specific requirements of the medication to be delivered or the prescription thereof.

### CROSS REFERENCE TO RELATED APPLICATIONS

This application claims the benefit of priority to U.S. Provisional Patent Application Nos. 60/736,802, filed 15 Nov. 2005, and 60/719,432, filed on 21 Sep. 2005, both of which, where permitted, are incorporated herein by reference in their entirety.

### FIELD OF THE INVENTION

Aspects of the present invention relate generally to validating (e.g., authorizing) and optimizing medicament delivery, or delivery of other agents such as nutritional agents, using medicament delivery devices, and more particularly to novel systems and methods comprising intelligent delivery devices (e.g., particle generation and dispersion devices, atomizers, nebulizers) having substantial utility for validating medicament use and/or optimizing medicament delivery parameters and/or storage and communication of medicament delivery related data.

### BACKGROUND

Increasing emphasis is being placed on assuring that medical devices used to deliver medication to patients operate consistently, and within parameters suited to specific requirements of the medications they are intended/validated to deliver. Currently, however, there are inadequate systems and methods for providing such assurances.

There is, therefore, a pronounced need in the art for novel and effective systems and methods for validating medicament use, and optimizing and/or customizing medicament delivery for medicament delivery devices.

### SUMMARY OF EXEMPLARY ASPECTS

Aspects of the present invention provide a smart device for validating medicament use and optimizing medicament delivery, comprising: a housing having an aerosolization chamber in which a medicament is aerosolizable, and having, or in communication with a holding means suitable for holding a medicament to be aerosolized; particle generation means in communication with the aerosolization chamber; an intelligent interface comprising an input sensing or acquisition means suitable to acquire information from a medicament-associated ID tag, and operative with the intelligent interface to provide for validated medicament delivery, and to provide for at least one of optimization of the particle generating means, and setting of a delivery dosage control parameter, based on the information acquired from the medicament-associated ID tag; and a user adapter in communication with the aerosolization chamber, the adaptor configured to facilitate delivery of a validated medicament to a user. In particular implementations, the device further comprises at least one particle dispersion chamber integral to, or in communication with the aerosolization chamber and the user adapter, to provide for optimized delivery of aerosolized particles to a user.

Additional exemplary aspects of the present invention, provide a method for validating medicament use and optimizing medicament delivery, using a particle generation and delivery device, comprising: providing user data by an end-user to a prescribing entity or physician to provide for a prescription for a medicament; providing the prescription of the prescribing entity or the physician to a prescription drug supplier or pharmacy, the prescription comprising user authorization for use of the prescription medicament by the end-user; providing, by a medicament supplier, the medicament and associated medicament data to the prescription drug supplier or pharmacy; providing, by a device supplier, a particle generation and delivery device along with a list of medicaments validated for delivery by the device; providing device parameters suitable to optimize particle generation and delivery of the prescription medicament by the particle generation and delivery device; inputting the medicament data and device parameters, and adjusting the particle generation and delivery device according to the device parameters; recognizing, by the device, the prescription medicament as a validated medicament; and delivering, using the adjusted particle generation and delivery device, of the validated medicament to the end-user.

Further exemplary aspects of the present invention provide a computer implemented method for validating medicament use by, and optimizing medicament delivery to an end-user, comprising: configuring, in one or a plurality of electronic databases stored in a storage device of a computerized particle generation and delivery device, a set of medicament validation data for medicaments authorized for delivery by the device, and a set of device parameters suitable to optimize particle generation and delivery of a validated medicament by the particle generation and delivery device; inputting medicament associated information from a medicament ID tag into the device; validating, using a software program stored on the storage device that is operative with a processor of the computer to receive and process the medicament associated information to provide a deliverable validated medicament, and is operative to provide for optimizing the particle generation and delivery device according to the device parameters; and delivering, using the optimized device, the validated medicament to an end-user. In particular aspects, the set of device parameters suitable to optimize particle generation and delivery of a validated medicament by the particle generation and delivery device is input in to the device along with the medicament associated information.

### BRIEF DESCRIPTION OF THE DRAWINGS

FIGS. 1A and 1B show a flow chart for a method of medicament recognition and system setup.

FIG. 2 is a flow chart for system operation.

FIG. 3 is a flow chart for caretaker authorization.

FIG. 4 is a flow chart for system communication.

FIG. 5 is a flow chart for system cleaning.

FIG. 6 is a flow chart for dose administration.

FIG. 7 is a flow chart for date/time system programming.

FIG. 8 is a block diagram of a controlled medicament delivery system.

FIG. 9 is an isometric view of an implementation of the delivery system.

FIG. 10 is a front elevational view of the implementation of the delivery system of FIG. 9.

FIG. 11 is a side-elevational cross-sectional view of the implementation of the delivery system of FIG. 9.

FIG. 12 is a side-elevational view of the implementation of the delivery system of FIG. 9.

FIG. 13 is a side-elevational cross-sectional view of a first implementation of the medicament output of the system of FIG. 8.

FIG. 14 is a side-elevational cross-sectional view of a second implementation of the medicament output of the system of FIG. 8.

FIG. 15 is a side-elevational cross-sectional view of a third implementation of the medicament output of the system of FIG. 8.

FIG. 16 is a side-elevational cross-sectional view of a fourth implementation of the medicament output of the system of FIG. 8.

FIG. 17 is an event diagram of a first exemplary scenario involving the system.

FIG. 18 is an event diagram of a second exemplary scenario involving the system.

FIG. 19 is an event diagram of a third exemplary scenario involving the system.

FIG. 20 is an event diagram of a fourth exemplary scenario involving the system.

### DETAILED DESCRIPTION

Particular aspects relate to intelligent devices for administration of therapeutic agents, such as medicaments, nutritional agents, and other agents and to provide novel systems and methods comprising intelligent delivery devices having substantial utility for validating medicament use, optimizing medicament delivery parameters, storage and communication of medicament delivery related data, etc., based on recognition and processing of identifying information associated with the medicament and/or packaging thereof. The systems and methods are broadly applicable to medicament delivery applications (e.g., respiratory tract, lungs, nasal passages and sinuses, eyes, etc) and devices (e.g., nebulizers, atomizers, particle generation and dispersion devices, etc.).

Exemplary delivery devices (e.g., devices for delivery of medicament to the respiratory system including the nasal, nasopharynx, and/or pulmonary systems) are designed to recognize and process identifying information associated with specific medications, and based thereon set operating parameters of the delivery device for optimal and/or customized delivery of the medication to a patient specifically to meet the needs of the medication and/or a prescription. In particular aspects the delivery device will not operate absent recognition of a valid medication intended for use in the delivery device. Further aspects provide additional features, including but not limited to information storage (e.g., information on how and when the medication was delivered, etc.), and communication of such information to one or more selected and/or authorized recipients.

Particular preferred aspects provide an interface between a medicament cartridge and/or its package and an electronic atomizer/nebulizer used to deliver a medication to a patient. An intelligent ID tag (e.g., RFID tag, magnetic strip, bar code etc.) is imbedded within, or otherwise operatively associated with a medicament cartridge/package, and is recognizable or readable by a corresponding electronic sensing means located in, or associated with the atomizer/nebulizer (or where information on the ID tag is readable for input into a sensing or reading or input means), whereby ‘validation’ (e.g., authorization, activation of the medicament or particle delivery function of the device) is afforded allowing the atomizer/nebulizer to operate, and preferably setup or configured to operate optimally, or in a customized fashion, in view of specific requirements of the medication to be delivered and/or the user.

In particular aspects, once a medicament (e.g., pharmaceutical composition) is recognized and accepted (e.g., validated), the intelligent device will set a ‘go’ or ‘no-go’ ‘flag’ in the system that will either allow it to operate or not. In preferred aspects, a variety of intelligent operating functions and/or parameters are coordinated to vary or optimize the operating characteristics of the delivery device and/or set controls (e.g., limitations) on, for example, how, how much and/or when the medicament is delivered to the patient. In certain aspects, the usage data on the unit operation is stored, and is optionally made available to any one or more of a number of responsible authorized parties to, for example, monitor and manage the appropriate administration of the medication.

Medicament Recognition—In particular aspects, a medicament delivery device (e.g., a particle aerosolization device with a user adapter (oral, nasal ocular, etc.), a particle generation and delivery device with a user adapter, an atomizer or a nebulizer optionally having, along with a user adapter, one or a pair of particle dispersion chambers to provide delivery of dispersed (e.g. vortical/turbulent flow) particles into one or both nostrils of a user, such as ViaNase® device, or another device for delivery of medicament to some aspect of the respiratory system) comprises intelligent means (e.g., electronic, software, etc.) to interface with (e.g., recognize) specific medicament information associated with (e.g., delivered with, attached to, embedded within, integral with) the agent, nutrient or medicament package or unit dose ampoule (UDA). For example, exemplary interfaces may comprise RFID tags, Smartcards, Barcodes, keyboard entry, voice entry, network interface, modem interface, other electromagnetic interface, etc.

For example, the delivery device, by means of the interface, obtains particular medicament-specific information (e.g., the product ID, expiration date, specific package pedigree, etc.), processes this information (e.g., conducts internal analyses of such information), and sets operational device parameters based on them for optimal or customized medicament delivery.

Control Features. Exemplary parameters include, but are not limited to particular control features, such as: overall control (e.g., On/Off control); lockout (e.g., the device will not turn on if an incorrect product ID is recognized, or if the medicament/composition has passed its expiration date, if a specified elapsed time has not passed); caretaker control (the device will only turn on if the device is provided with an appropriate caretaker ID tag (a secondary ID tag of a caretaker) by a caretaker (e.g., parent, guardian, responsible caregiver, etc.).

Additional control features include operational parameter controls; variable operating parameters that are set based on medicament recognition/validation. Examples of such parameters include, but are not limited to: pump speed (e.g., varied to control droplet size and or atomization rate); compressed fluid pressure; pump pulsation or mode/pattern; compressed fluid pressure to particle dispersion chamber(s); variation in relative configuration/orientation and/or distance between a compressed fluid orifice and a liquid feed orifice of a Venturi-type nebulizer or atomizer particle generation means; adjustment of ‘vortical’ or turbulent flow properties; adjustment of breath activation feature; activation of cleaning cycle controls; etc.

Additional control features include medicament dose controls, including but not limited to: setting specific atomization time (e.g., in min per day, or number of times per day); setting of medicament prescription parameters (e.g., setting of specific number of times device can be operated. (e.g., 30 times for a 30 day supply in the prescription)); setting of alarm in the unit to encourage proper dosing (e.g., to beep when it is time to use the device); setting of alarm that reminds the user to clean the device (e.g., that goes off if the device is not separated after usage); etc.

Additional control features include operational information storage, including but not limited to information relating to: ID of medicament used; dose; time of day and date administered; length of atomization run (e.g., dose delivered); device operating operational parameters (e.g., as listed above); etc.

Additional control features include data communication aspects, including but not limited to: displays or lights; output to, for example, a smart card, etc.; downloads to PDA or PC devices; output to removable data cartridge, etc.

Data usage can be for a variety of purposes, including but not limited to: provision of clinical data for clinical trials to CRO or company; provision of data to a physician for support with compliance, etc.; provision of data to a pharmacy for prescription purposes (e.g., refills, etc.); etc.

### PREFERRED EXEMPLARY EMBODIMENTS

Aspects of the present invention provide a smart device for validating medicament use and optimizing medicament delivery, comprising: a housing having an aerosolization chamber in which a medicament is aerosolizable, and having, or in communication with a holding means suitable for holding a medicament to be aerosolized; particle generation means in communication with the aerosolization chamber; an intelligent interface comprising an input sensing or acquisition means suitable to acquire information from a medicament-associated ID tag, and operative with the intelligent interface to provide for validated medicament delivery, and to provide for at least one of optimization of the particle generating means, and setting of a delivery dosage control parameter, based on the information acquired from the medicament-associated ID tag; and a user adapter in communication with the aerosolization chamber, the adaptor configured to facilitate delivery of a validated medicament to a user.

In particular implementations, the device further comprises at least one particle dispersion chamber integral to, or in communication with the aerosolization chamber and the user adapter, to provide for optimized delivery of aerosolized particles to a user. In certain embodiments, the user adapter comprises a nasal, oral, or ocular adapter. Preferably, the adapter is a nasal adapter. In particular aspects, the intelligent interface comprises: a processor and at least one storage device connected thereto, the storage device comprising a stored set of validated medicament identifiers; and at least one stored software program operative with the processor to receive and process the information from the medicament-associated ID tag, to provide for validated medicament use, and to provide for at least one of optimization of the particle generating means, and setting of a delivery dosage control parameter, based on the information acquired from the medicament-associated ID tag. In certain embodiments, acquiring input information from the medicament-associated ID tag comprises use of at least one of RFID tags, smartcards, barcodes, keyboard entry, voice entry, network interface input, modem interface input, and wireless interface input. In particular embodiments, medicament use validation comprises: inputting information from a medicament ID tag; comparing the input medicament ID tag-associated information with a set of validated medicament identifiers stored in the at least one storage device of the smart device; and validating or not validating delivery of the medicament, based, at least in part, on the comparison. In certain aspects, validating or not validating delivery comprises recognition of the presence or absence of a correct medicament product ID. In certain embodiments, validating or not validating delivery comprises recognition of the presence or absence of a non-expired expiration date of the medicament. In particular aspects, validating or not validating delivery comprises recognition of the presence or absence of a sufficient period of lapsed time since the last medicament delivery. In certain aspects, validating or not validating delivery comprises recognition of the presence or absence of an inputted caretaker control key required for delivery of a medicament by the device to a user requiring caretaker authorization or assistance to receive the medicament. In particular aspects, optimization of the particle generating means based on the information acquired from the medicament-associated ID tag comprises adjusting operational parameters selected from the group consisting of: run time; atomization or nebulization rate; generated particle size; linear velocity of the particle; compressor pump output or pulsation pattern; compressor pump speed; compression fluid (e.g., air) pressure; vorticity velocity vectors or vortical flow characteristics; turbulent flow characteristics; dispersion chamber switching or activation/deactivation; variation in relative configuration/orientation and/or distance between a compressed fluid orifice and a liquid feed orifice of a Venturi-type nebulizer or atomizer particle generation means; and combinations thereof. In certain embodiment, the at least one stored software program operative with the processor to receive and process the information from the medicament-associated ID tag, additionally provides for a caretaker control feature, wherein input of a caretaker control key is required for delivery of medicament by the device to a user requiring caretaker authorization or assistance to receive the medicament. In certain aspects, the at least one stored software program operative with the processor to receive and process the information from the medicament-associated ID tag, additionally provides for storage of a set of historical operational information data in the at least one storage device and further provides for data communication or transmission of the stored historical usage data to PC or PDA devices, smart cards, removable data cartridges, or to one or more authorized or responsible recipients to monitor or manage medicament prescription or administration.

Additional exemplary aspects of the present invention, provide a method for validating medicament use and optimizing medicament delivery, using a particle generation and delivery device, comprising: providing user data by an end-user to a prescribing entity or physician to provide for a prescription for a medicament; providing the prescription of the prescribing entity or the physician to a prescription drug supplier or pharmacy, the prescription comprising user authorization for use of the prescription medicament by the end-user; providing, by a medicament supplier, the medicament and associated medicament data to the prescription drug supplier or pharmacy; providing, by a device supplier, a particle generation and delivery device along with a list of medicaments validated for delivery by the device; providing device parameters suitable to optimize particle generation and delivery of the prescription medicament by the particle generation and delivery device; inputting the medicament data and device parameters, and adjusting the particle generation and delivery device according to the device parameters; recognizing, by the device, the prescription medicament as a validated medicament; and delivering, using the adjusted particle generation and delivery device, of the validated medicament to the end-user. In particular aspects, the particle generation and delivery device is adjusted according to the device parameters by the prescription drug supplier or pharmacy, and thereafter provided by the prescription drug supplier or pharmacy to the end-user along with the prescription medicament. In certain aspects, the device parameters are provided to the prescription drug supplier or pharmacy by the device supplier, by the medicament supplier, or by the prescribing entity or the physician. In certain embodiments, the prescription further comprises a caretaker authorization code or key that must be input into the device to allow for delivery of the validated medicament. In certain aspects, the medicament data is printed and/or electronic, and comprises at lest one of medicament identification, expiration, pedigree or e-pedigree, and user instructions. In certain aspects, delivering, using the adjusted particle generation and delivery device, of the validated medicament to the end-user comprises: delivery of an initially authorized sub-prescription number of doses that is less than the total number of doses for the associated prescription; requesting, by the end-user, authorization for additional doses from prescribing entity or the physician; obtaining said authorization; and delivery of said additional doses to the end-user. In certain implementations, at least one of: providing user data by an end-user to a prescribing entity or physician to provide for a prescription for a medicament; providing the prescription of the prescribing entity or the physician to a prescription drug supplier or pharmacy, the prescription comprising user authorization for use of the prescription medicament by the end-user; providing, by a medicament supplier, the associated medicament data to the prescription drug supplier or pharmacy; providing, by a device supplier, a list of medicaments validated for delivery by the device; providing device parameters suitable to optimize particle generation and delivery of the prescription medicament by the particle generation and delivery device; inputting the medicament data and device parameters, and adjusting the particle generation and delivery device according to the device parameters; and recognizing, by the device, the prescription medicament as a validated medicament, comprises transmission of data over a local area network (LAN), wide area network (WAN), or wireless network.

Further exemplary aspects of the present invention provide a computer implemented method for validating medicament use by, and optimizing medicament delivery to an end-user, comprising: configuring, in one or a plurality of electronic databases stored in a storage device of a computerized particle generation and delivery device, a set of medicament validation data for medicaments authorized for delivery by the device, and a set of device parameters suitable to optimize particle generation and delivery of a validated medicament by the particle generation and delivery device; inputting medicament associated information from a medicament ID tag into the device; validating, using a software program stored on the storage device that is operative with a processor of the computer to receive and process the medicament associated information to provide a deliverable validated medicament, and is operative to provide for optimizing the particle generation and delivery device according to the device parameters; and delivering, using the optimized device, the validated medicament to an end-user. In particular aspects, the set of device parameters suitable to optimize particle generation and delivery of a validated medicament by the particle generation and delivery device is input in to the device along with the medicament associated information. In certain embodiments, the medicament associated information and the device parameters are both part of the medicament ID tag. In particular preferred embodiments, the medicament is a prescription medicament, and the medicament ID tag is attached, imbedded, integral to, or otherwise associated with the prescription medicament to provide for validated medicament use and optimal delivery thereof. In certain implementations, the particle generation and delivery device is provided to the end-user along with the prescription medicament.

Certain Aspects are Illustrated by the Following Description of the Figures.

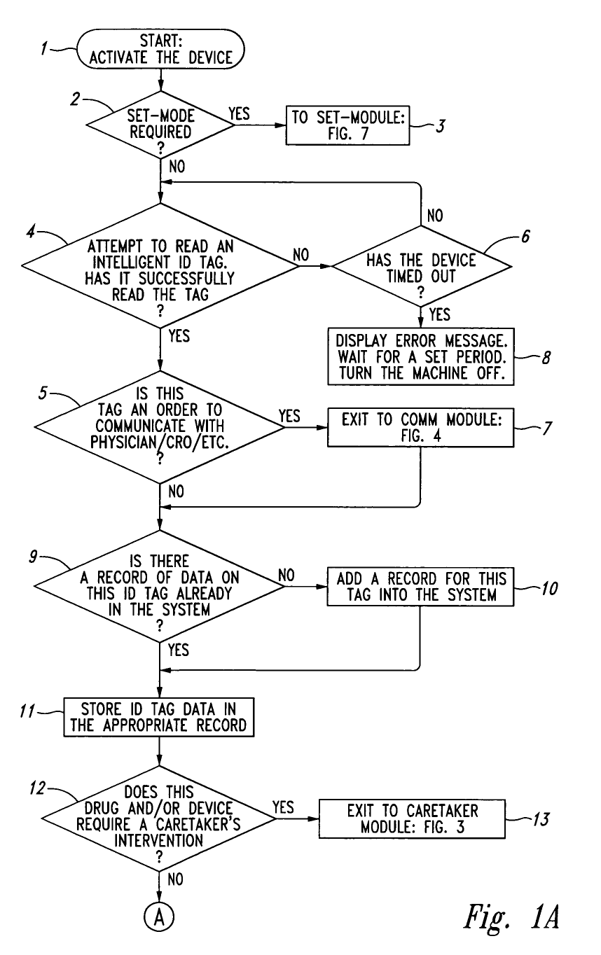

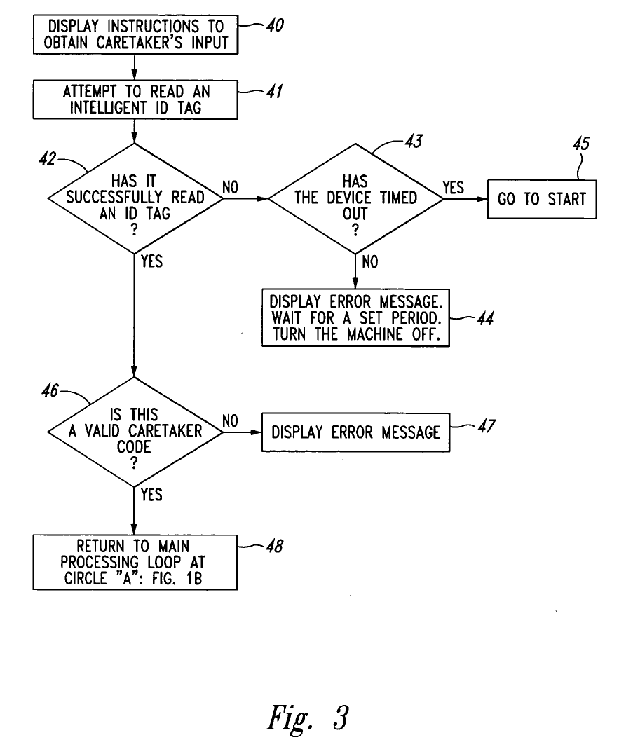

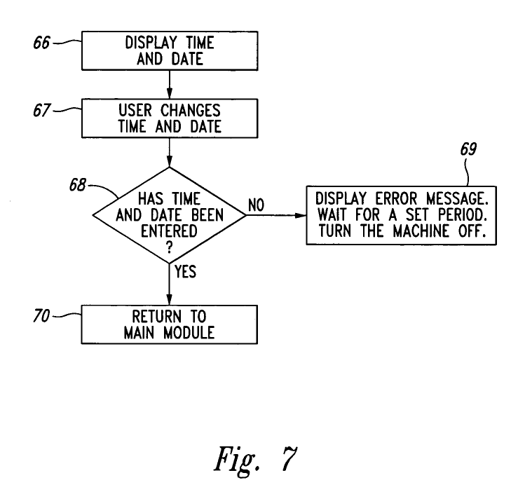

FIGS. 1A and 1B show an exemplary flow chart of Medicament Recognition and the Set up of an implementation of the device prior to operation. As seen in FIG. 1, a user (e.g., a patient) activates the device 1. This is accomplished with an on/off switch. The user reviews the display on the unit to determine if it is displaying the correct date and time, or if there is no date or time to be displayed (set-Mode not required). If the date and or time is incorrect, the user changes the data (FIG. 7). After accomplishing the date and or time change, the information of the ID tag is read (e.g., a user positions the intelligent ID tag in close proximity to the device and presses the “Read Tag” button 4; a user reads information from the tag and inputs the information into the device; or a device read function automatically activates (e.g., for some length of time) upon turn-on of the device). If the tag comprises an ‘order to communicate,’ the user/patient/caretaker accomplishes that 7 communication (FIG. 4). If there is no such order to communicate, the system software compares the data on the ID tag to that stored in memory 9. If the data from the ID tag, or some portion thereof (e.g., prescription data, expiration date, etc.) is not in the data base, the data is added 10. Data from the ID tag is added to an appropriate or correct location in memory 11.

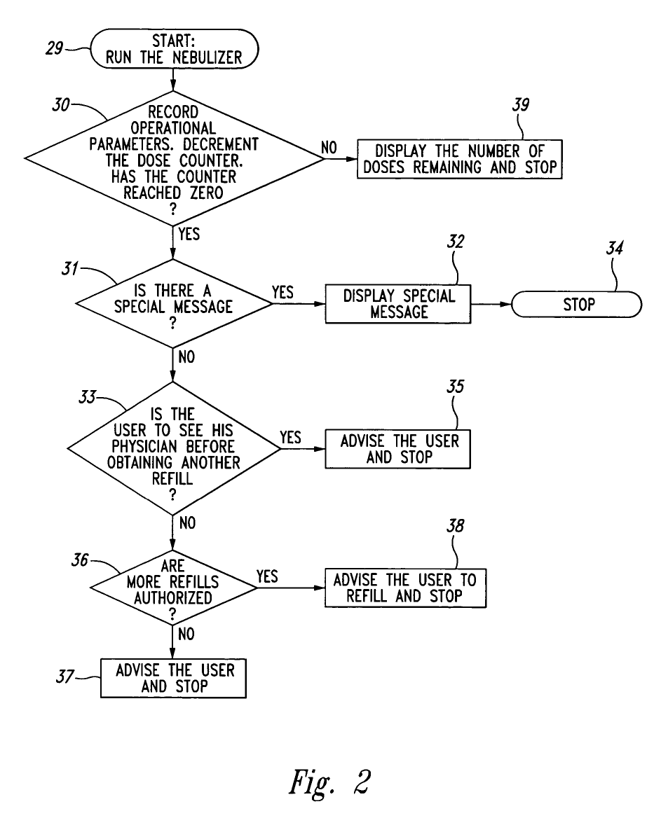

A provision is included to permit a “caretaker” to control further delivery of medication by the device based on their instructions from a physician or other responsible authority. This will permit the control of the use of the device for children and or particular elderly and serves as another control to limit unauthorized use of the device. In this situation the caretaker has an intelligent ID tag that must be recognized by the device in addition to the ID tag of the medicament. In other implementations intelligent ID tags are used for this and/or other data transmission purposes such as for medicament ID, pedigree information, administration data, other authorization data, device parameter data, physician or pharmacist identification, etc. The device will recognize whether or not a caretaker approval is required 12. If it is required, Caretaker Authorization is accomplished FIG. 3.

Once the Caretaker Authorization is accomplished, or if it is not required, the software program compares the data on the medicament intelligent ID tag with authorization/validation data (e.g., validated medicament identifier data for agents, solutions, medicaments, etc., for which the device is authorized to deliver) stored in memory 14. If the comparison supports authorization/validation (e.g., the information on the ID tag conforms to authorization/validation data, for example, conforms as to product code, expiration date, etc. the agent, solution, nutrient, medicament, etc., is declared valid for use 14 and the delivery and or further validation process proceeds. If the data does not conform, the message on the display indicates the agent, solution, nutrient, medicament, etc., is not valid for use and the device turns off 15. In particular embodiments the device authorization/validation information (that is compared with information on the agent, medicament ID tag) comes pre-stored in the device. Alternatively, the authorization/validation information can be input by a user or other authorized person having particular code or key means to input authorization/validation information into the device. Alternatively, the authorization/validation information can be updated (e.g., by placing particular additional codes or keys on the agent/medicament ID tag, such that the device is updated to validate use for a medicament upon reading the corresponding medicament ID tag.

If the data in memory for the medicament indicates that a check is to be made to insure that the medicament has not passed its expiration date, a clock function will be enabled. This is checked at 16. If it is enabled, the expiration date from the data base in memory, or the intelligent ID tag is compared with the current date 17. If the date has passed, a message to indicate such is displayed and the unit is turned off. If the expiration date has not passed, the unit continues to operate.

If the data in memory for the medicament indicates that the medicament may only be used after a specific time interval, a “lock-out” time feature will be enabled. If this is enabled for the specific medicament to be administered, the device will make sure the appropriate amount of time has passed before the dose can be administered/taken 20. If the time interval has not been exceeded, the device will indicate that the dose can not be taken 24. If there is no lock-out time interval for the medicament, the unit continues to operate.

The intelligent ID tag may contain information on the number of doses permitted, for example, by a prescription. This information may also be stored in unit memory. As the number of doses is counted by the system, the number can be compared to the prescribed number of doses. This is compared 21 for a remaining dose left on the prescription. If there are no more doses permitted by the prescription, 22 a message is displayed and the unit will stop 24. The software may also identify whether refills of the prescription are permitted 23, and if they are permitted, a message is displayed to advise the patient to obtain a refill 25 or see his physician 26.

If there is at least one dose remaining on the counter 21, the process continues and the logic in memory questions if there are any special operational parameters to be set to have the delivery device operate to meet the specific requirements of the medicament to be administered 27. If special parameters such as run time, droplet size, droplet velocity, pump pulsation, etc. are required they will be set for the operation of the device 28.

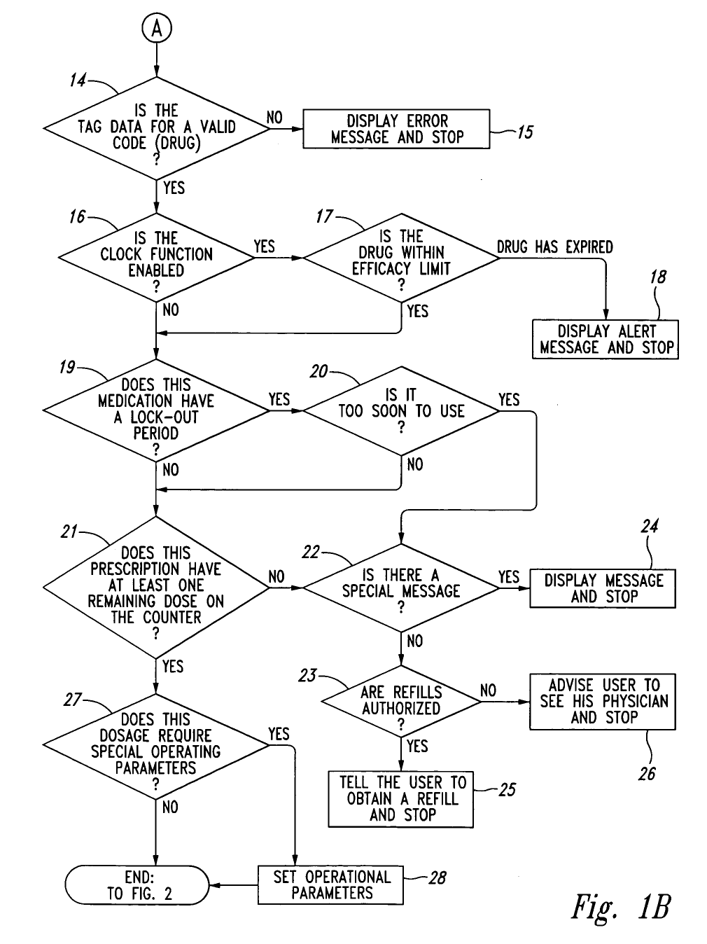

FIG. 2 shows a flow chart of the running of the exemplary nasal delivery device. A liquid medicament from the unit dose ampoule is emptied into a medicine chamber of the device, and the user inhales the aerosolized medication into at least one nasal cavity 29. During this time the operational parameters set 28 previously are monitored, and recorded in the memory. This data is used later for analysis and control of the device and/or for authorized reporting/usage data transmission.

Once the dose has been administered, the dose counter in the unit memory is reduced by one 30. If the dose counter has reached 0, and message is displayed to indicate same to the patient 32 and the unit stops operation 34. If there is a requirement to see the responsible physician prior to obtaining another refill of the prescription 33, the user will be advised 35. Otherwise (NO branch of decision step 33) the system determines whether more refills are authorized 36, and if so, advises the user to stop 37. Otherwise, (NO branch of decision step 36), the user is advised to refill and stop 38. At the end of the administration of the dose, the remaining number of doses will be indicated on the display 39.

FIG. 3 shows a flow chart of an exemplary Caretaker Authorization Module. It is accomplished, if required, at step 12 above (FIG. 1A). Once in this loop, the device will indicate that a Caretaker's input is required 40. The caretaker will place his or her intelligent ID tag in close proximity of the device and press the Read Tag button 41 (or otherwise input (e.g., keyboard entry, download, etc) a suitable Caretaker authorization code or key into the device). If the Caretaker tag or Caretaker input information is correctly matched to the data base information 46, the process will return to the main FIG. 1 processing loop at step 14. If the caretaker code is not valid when compared to the memory, the unit will display an error message to indicate such 41.

FIG. 4 shows a flow chart covering the Communication Module. The device is capable of communicating with a variety of devices such as a Personal Computer (PC), Docking Station, Removable Storage device, etc 49. This communication is used to download the history of dosing, operational parameters, and other data that may be required to assist in management of a medical condition, analysis of clinical trial information, review of operation of the unit, etc. The unit will sense when a connection 50 with the appropriate communication device. A successful linking is required 51. If the linking is successful, the data will be transmitted 54, a Success message will be displayed, and the unit will shut off 55. If the data is not transmitted successfully, an error message will be displayed 56, and the transmission will be tried again until successful 57.

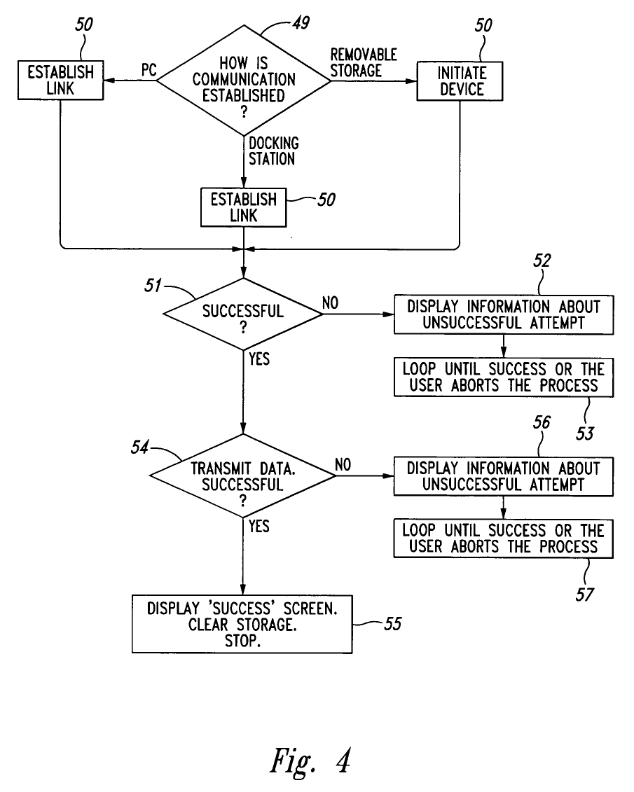

FIG. 5 shows a flow chart of the Cleaning Module. Once the delivery device has delivered a dose and has been operated for a set number of times (n=1 or greater), 58, a message is displayed indicating that cleaning of the device is required 59. If cleaning is not required 58 NO, no further processing is required and the loop ends 61.

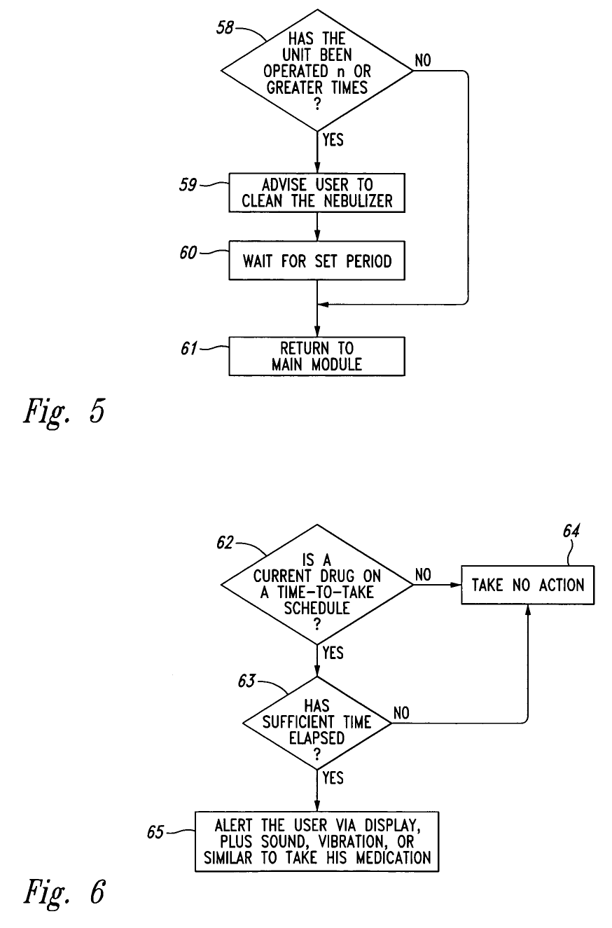

FIG. 6 shows a flow chart of the “Time-to-take” Module. If the medicament as recognized by the intelligent ID tag has a specific time-to-take a dose, the unit will recognize when it is time to take the dose 62. When the specific time has elapsed, or when the time on the timer indicates it is time to take the dose 63 a message is displayed and/or an audio or vibratory alarm is activated indicating such 65. If time has not elapsed, or the time has not arrived to take the dose no action is taken 64.

FIG. 7 shows a flow chart indicating the ability to set the date and time. Once the user observes that the date and or time is incorrect on the display 66, the user changes the time to the correct time 67 by an entry key. The unit will then loop to the main process at 4.

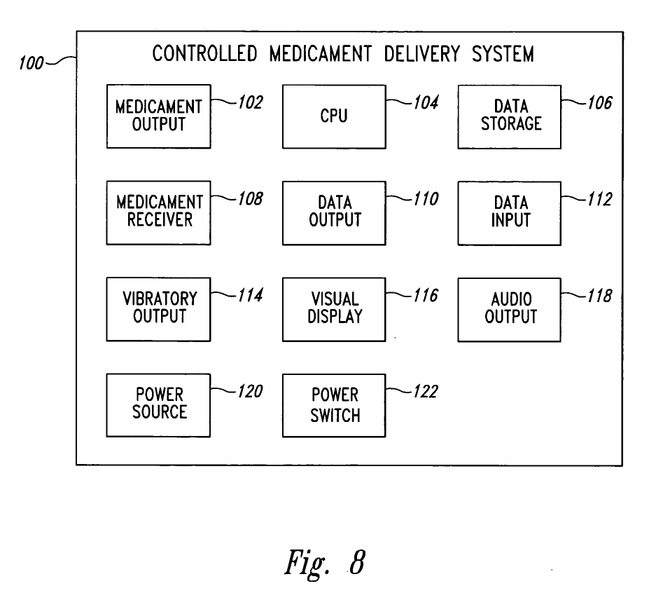

An exemplary controlled medicament delivery system such as that shown in FIG. 8, implements procedures and methods including those described in the flowcharts discussed above for FIGS. 1-7. The system includes a medicament output 102, a central processing unit (CPU) 104, a data storage 106, a medicament receiver 108, and data output 110, a data input 112, a vibratory output 114, a visual display 116, an audio output 118, a power source 120, and a power (on/off) switch 122.

An implementation of the medicament output 102 depicted herein is described below as an inventive nebulizer with particle dispersion chambers for votical/turbulent flow of particles into one or more nostrils. Parameters of the depicted medicament output 102 include duration of individual treatment session for dose administration (run time), droplet size of dispersed particle, droplet linear velocity at exit point from the system 100, pump pulsation pattern, pump speed, compressed fluid (e.g., air) pressure, details regarding left to right chamber switching or chamber activation, vorticity velocity, turbulent flow characteristics, relative configuration/orientation (in the context of Venturi-type atomization or nebulization means) of, or distance between a compressed fluid orifice and a liquid feed orifice, etc.

The CPU 104, among other things, sets these and other parameters for the medicament output 102 based upon data from the data storage 106 and/or the data input 112 and/or the medicament receiver. Parameter data can be initially inputted into the system 100 through the data input 112 at time of assembly of the system or can be later inputted into the system through either the data input 112 or the medicament receiver 108. The CPU 104 is represented as a single unit, however, in other implementations processing can be handled by a number of different units. The CPU 104 other aspects such as implementing methods such as those represented by the flow charts described above, including management of operational parameters, management of authorization and access control, on-going monitoring of system usage, and reporting on various activities involved with the system 100.

Data is generally stored in the data storage 106, which is represented as a single unit, but in other implementations the data storage can be handled by a plurality of data storage units. Data stored can include operational parameter data, user data, physician data such as identification, pharmacy data such as identification, medicament data such as origination data, tracking data (such as through a pedigree or e-pedigree system), medicament administration data (such as dosage schedule, use authorization schedule, user-specific instructions, etc.), etc.

In the depicted implementation, the medicament receiver 108 receives containers, such as ampoules, of medicament doses, such as unit doses. In some implementations, the medicament containers include data storage to indicate one or more aspects such as medicament identification, expiration, tracking such as through a pedigree or e-pedigree system, and parameter settings for the medicament output 102. The CPU 104 can send one or more portions of data from the medicament container to the data storage 106 for further use. In some implementations, the medicament container can also include other data such as physician identification data or prescription data either to be used with methods such as authorizing use or adjusting operational parameters for the medicament output 102.

The data output 110 can include one or more of the following forms depending upon how and to what extent data from the system 100 is supplied or stored. Generally data supplied from the system 100 could be in the form of status data to track operational performance, to track usage compliance by the user, to further understanding of effectiveness of a protocol, or other use. In some implementations, the data output 110 could be included in a magnetic strip connection for such data storage devices as a smart card, a modem connection, a computer connection such as a universal serial bus connection, a network connection, a wireless connection, a data storage device connection such as a memory chip connection, a printer connection, a monitor connection, an radio frequency identification (RFID) connection, or other connection. The data output 110 can include device hardware to communicate over such connections or other connections as well.

The data input 112 can include one or more of the following depending upon how and to what extent that the system 100 is initialized and updated thorough its lifecycle. Examples of the data input 112 include a magnetic strip reader to access such data storage devices as a smart card, a modem connection, a computer connection such as a universal serial bus connection, a network connection, a wireless connection, a data storage device connection such as a memory chip connection, an radio frequency identification (RFID) connection, a keypad connection, a barcode connection, or other connection. The data output 110 can include device hardware to communicate over such connections or other connections as well.

In some implementations, the CPU 104 can control the vibratory output 114 to indicate modes of operation such as “pulsating” or “steady flow,” side to side administration details, instructions such as “breath in through the nose and out through the mouth” or “breath normally,” and various status messages such as “your dose is complete.”

In some implementations, the CPU 104 can control a visual display 116 such as a display screen to output such data as medicament product name, time to next dose, clock indicating elapsed time as the dose is administered, modes of operation such as “pulsating” or “steady flow,” side to side administration details, instructions such as “breath in through the nose and out through the mouth” or “breath normally,” and various status messages such as “your dose is complete.” Other implementations use a visual display 116 that may include one or more indicator lights in addition to a display screen or instead of a display screen.

In some implementations, the CPU 104 can control an audio output 118 as a voice enabled speaker and in other implementations as a series of tones or other noises. Some implementations could announce such data as medicament product name, time to next dose, clock indicating elapsed time as the dose is administered, modes of operation such as “pulsating” or “steady flow,” side to side administration details, instructions such as “breath in through the nose and out through the mouth” or “breath normally,” and various status messages such as “your dose is complete.”

The power source 120 can be manually controlled through the power switch 122 or can also be controlled through the CPU 104 to implement aspects of methods such as those described above associated with the flow charts of FIGS. 1-7.

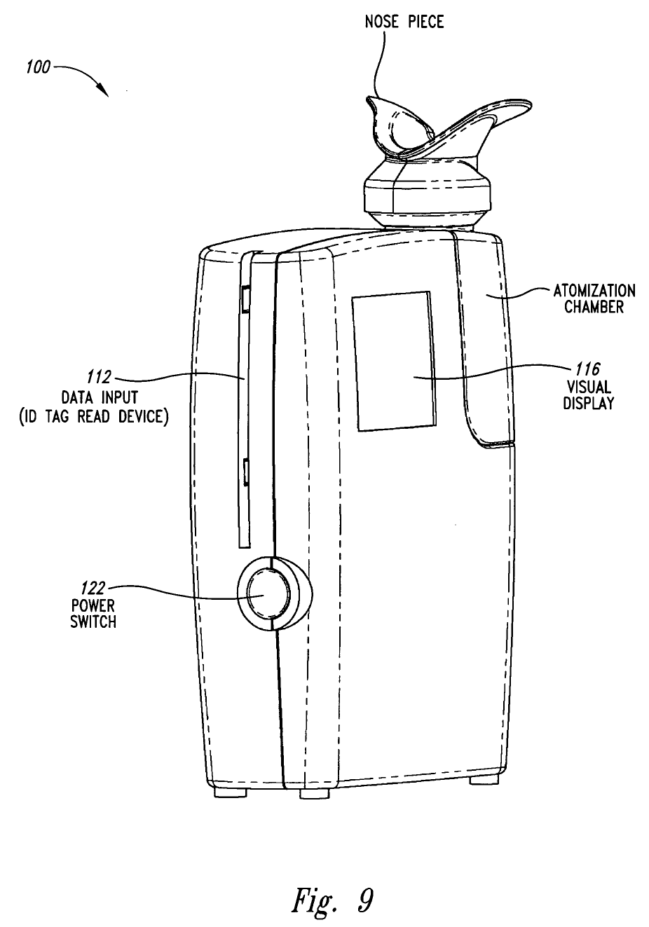

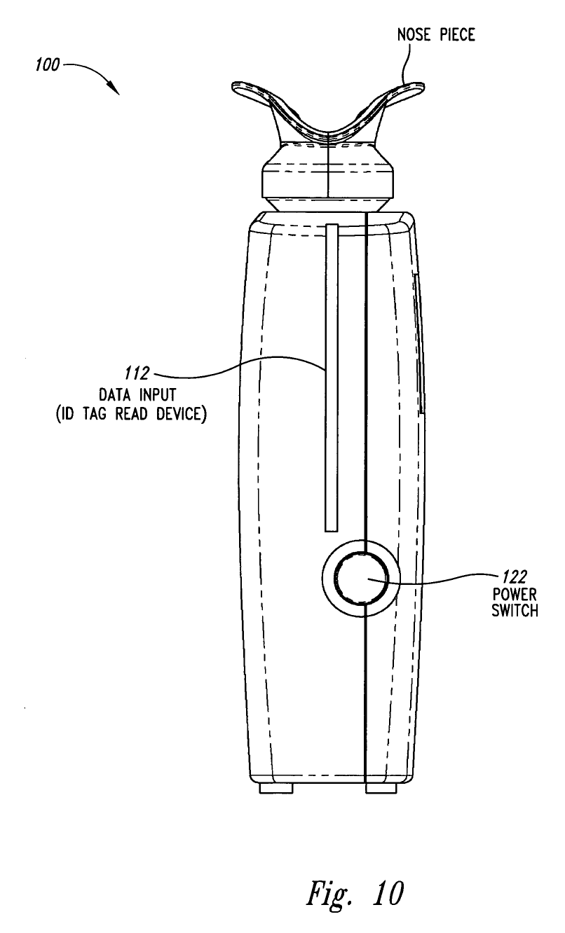

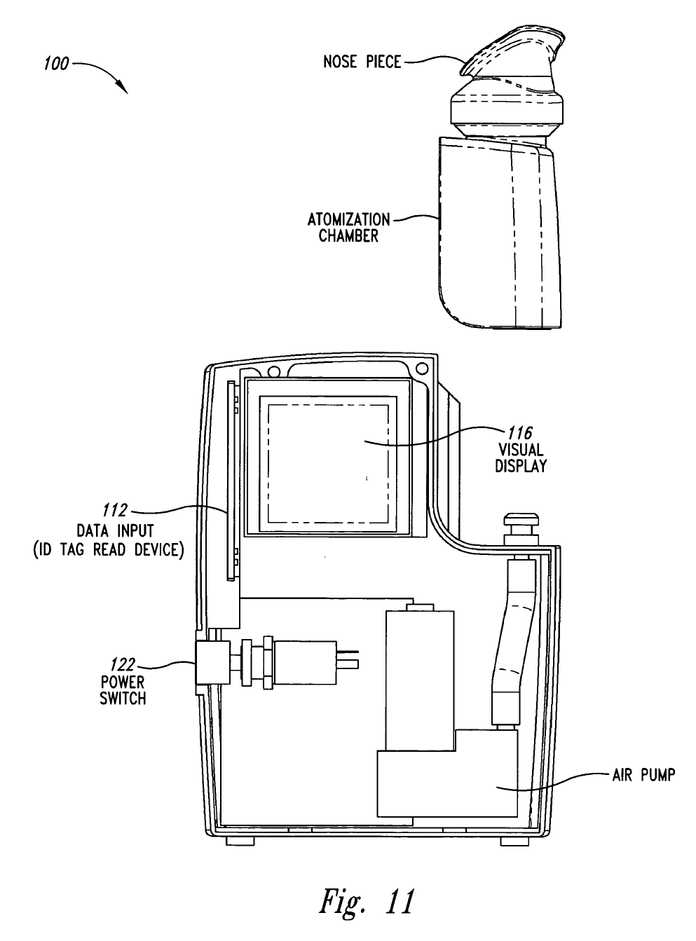

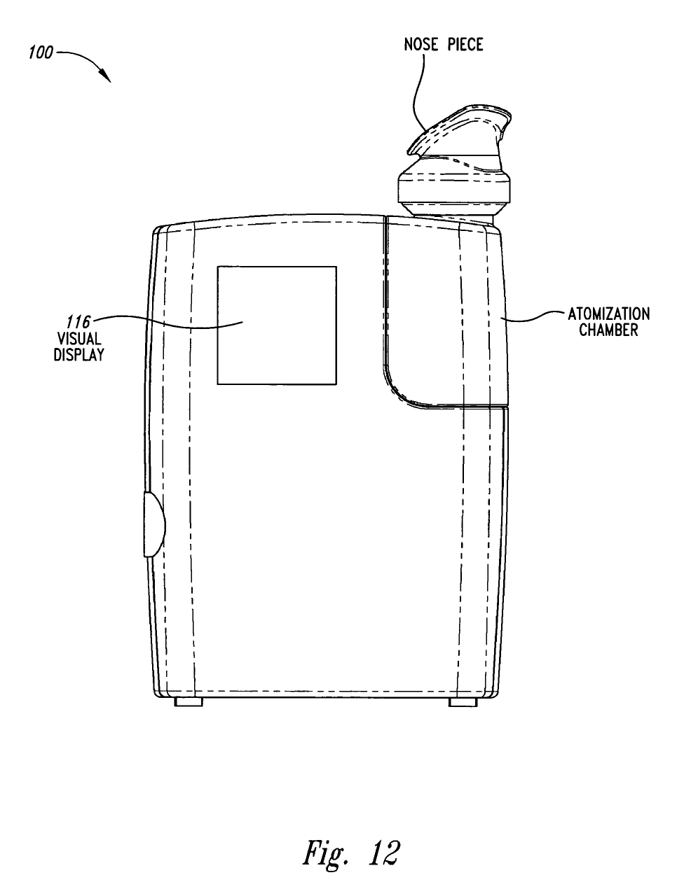

A depicted implementation of the system 100 is shown in FIGS. 9-12 as a version of a nebulizer with particle dispersion chambers and delivery of vortical or turbulent flow of dispersal particles into user nostrils. An instance of the visual display 116 and an instance of the data input 112 as an ID tag read device are shown in the depicted implementation.

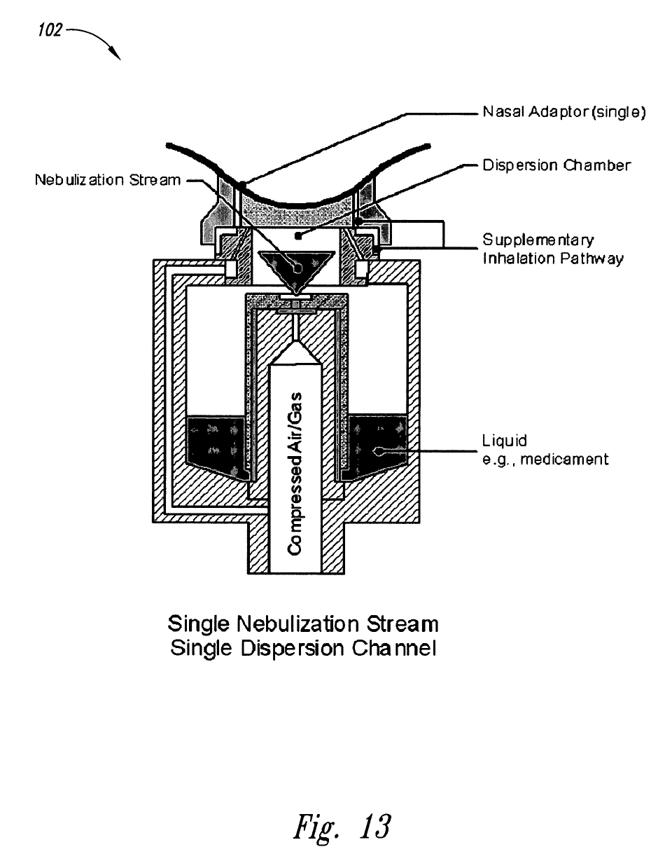

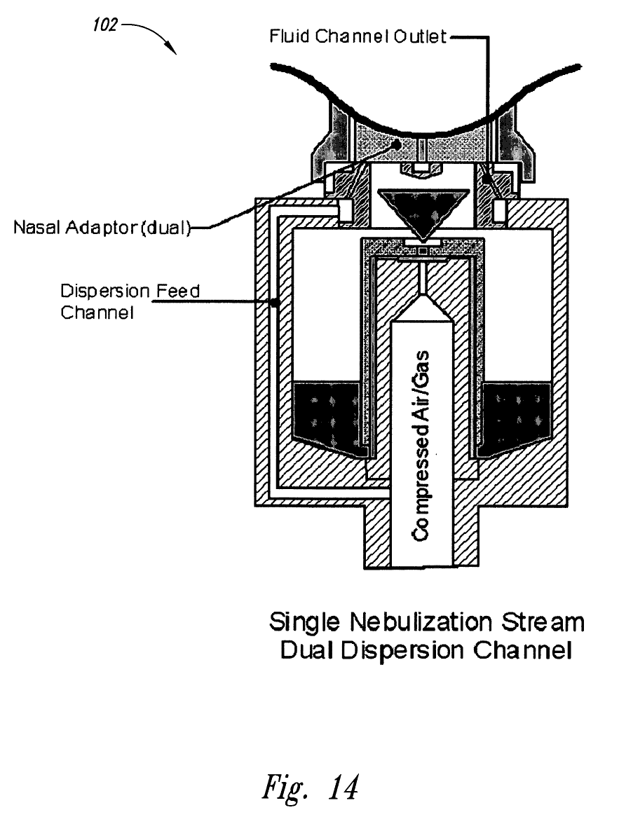

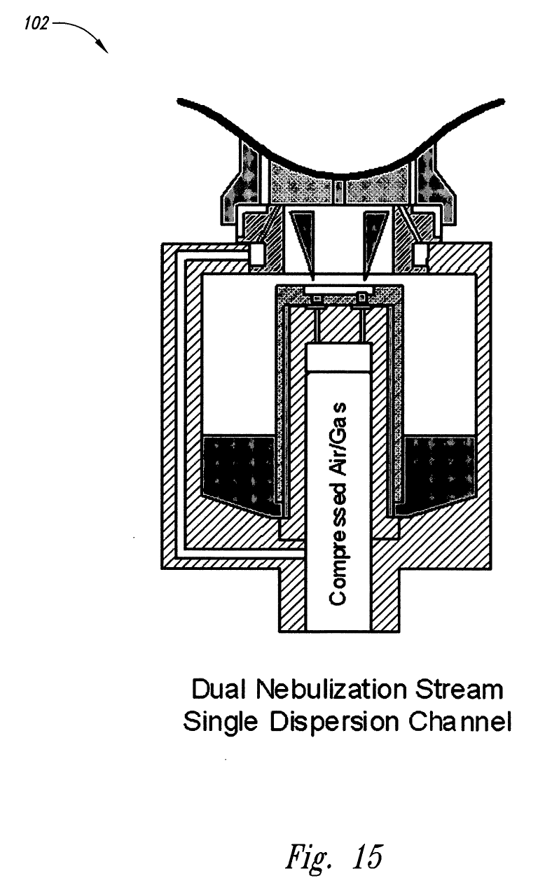

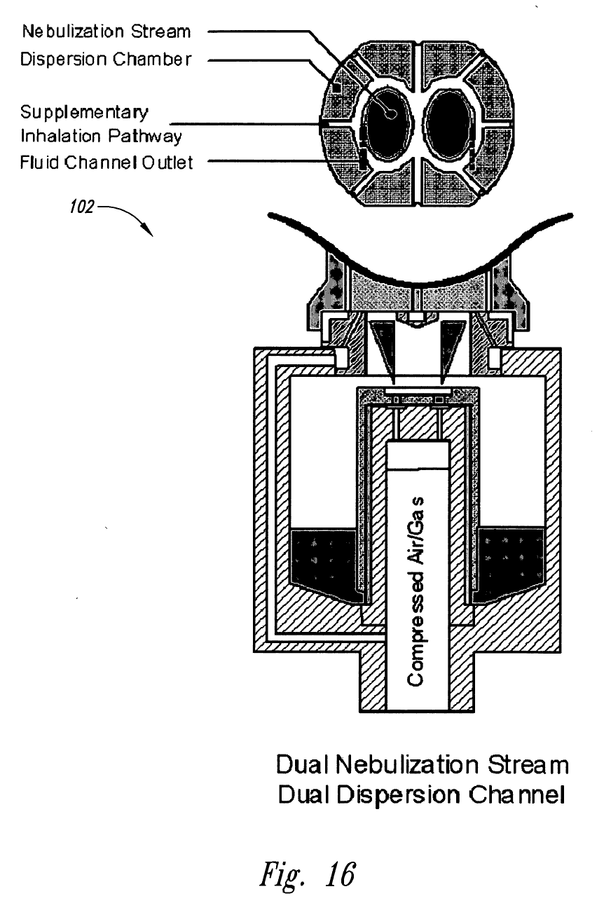

An implementation of the medicament output 102 is shown in FIGS. 13-16 as having a nebulizer (or alternatively an atomizer) being in communication with a pair of particle dispersion chambers (or with a pair of particle dispersion channels within one particle dispersion chamber) disoriented to provide direct parallel (or substantially parallel) delivery of vortical flow particles into each nostril via a complementary bifurcated nasal adapter. Such dual delivery significantly eliminates any medicament loss resulting from particle collisions with the center of the nose between the two separate nasal passages.

Additionally, dual particle dispersion channels allow for setting different vortical parameters (e.g., angle, velocity, direction, etc.) for each particle dispersion channel, and further allows for vortical flows having opposite directions (a preferred aspect). The dual design allows for a vortical flow to be targeted to each nostril, and the vortical flow is not interrupted by flow colliding with the division between the nostrils. Thus, the dispersion parameters can be optionally and uniquely tailored to individual users if necessary or desired (e.g., for long-term users treating chronic conditions, or where one nostril is relatively obstructed or otherwise distinguishable from the other.

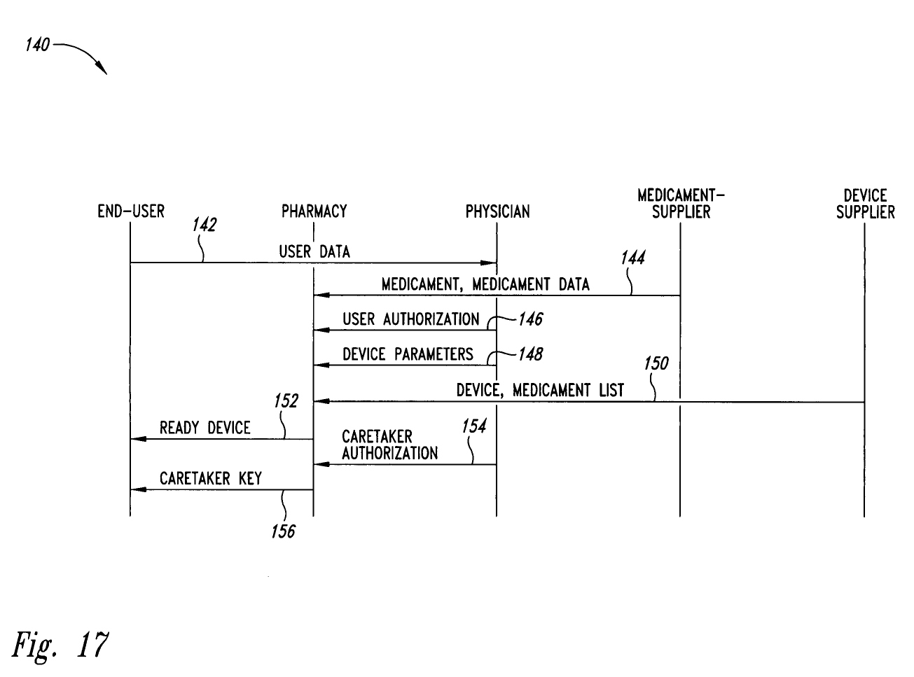

Some aspects of data usage associated with the system 100 are illustrated with a few depicted scenarios shown for illustrative purposes in FIGS. 17-20. In a first scenario 140 shown in FIG. 17, an end-user, such as a patient, provides user data (transmission 142) to a physician, typically in the physician's office. At a time before, after, and/or during the transmission 142, a medicament supplier sends medicament and medicament data to a pharmacy (transmission 144). The medicament data can include medicament identification, expiration, pedigree or e-pedigree, and user instructions. The medicament data can be printed and/or electronic. The data form will impact how the data is further handled as explained below.

After the user data is received by the physician (transmission 142), the physician sends user authorization to the pharmacy (transmission 146) and sends device parameters to the pharmacy (transmission 148) typically as part of a prescription that The pharmacy can then manually adjust the system 100 according to the device parameters if, for instance, the device parameters are in a printed form and/or the system 100 is only configured for manually adjustment. The pharmacy can input the device parameters electronically through the data input 112 if the device parameters are stored electronically stored and the system 100 is configured for electronic programming such as through the CPU 104 and the data storage 106.

At a time before, after, or during the previous transmissions described above, a device supplier sends an instance of the system 100 along with a medicament list to the pharmacy (transmission 150). In some implementations, the medicament list is stored electronically in the data storage 106 and is used by the CPU 104 for authorization procedures as to what medicaments are allowed to be used with the system 100.

Once the pharmacy has received the transmission 144, the transmission 146, the transmission 148, and the transmission 150, the pharmacy can deliver a ready device instance of the system 100 (transmission 152) to the end-user. A ready device instance of the system 100 in this depicted scenario 140 includes being adjusted regarding operational parameters for the prescribed medicament, having authorization for use of the medicament through use of the medicament list, and having the prescribed number of doses of the medicament.

In a situation in which the end-user is not of age or otherwise capable of self-administration of the medicament, a caretaker is designated by the physician through a caretaker authorization (transmission 154) sent to the pharmacy. The pharmacy then can issue a caretaker key to the end-user's caretaker (transmission 156). The caretaker key can be in the form of a data storage that can be read by the data input 112 and/or a password that can be hand keyed in through a keyboard instance of the data input 112 and/or through some other form of the data entry. The caretaker key provides to the system 100 sufficient identification data so that the system is notified of the presence of the caretaker and can be subsequently activated to be used by the end-user to receive a dose of medicament.

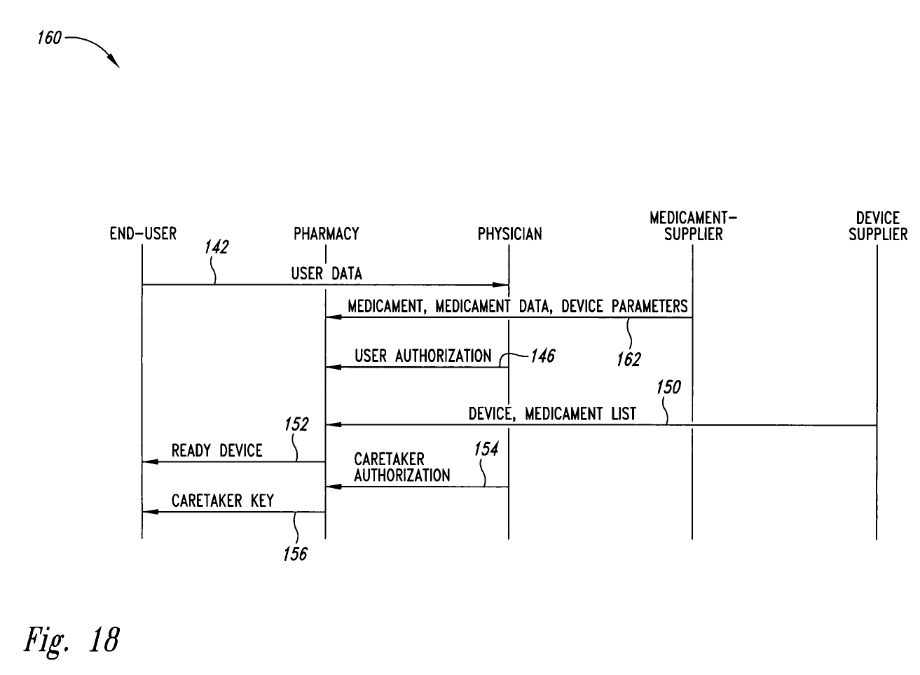

A second scenario 160 is shown in FIG. 18 in which the device parameters are sent from the medicament supplier to the pharmacy along with the medicament and medicament data (transmission 162). In this scenario, the device parameter data may be stored in the same or different form as the medicament data. For instance, the medicament data and the device parameters may be stored electronically in the same storage such as in a smart card to be read by the data input 112.

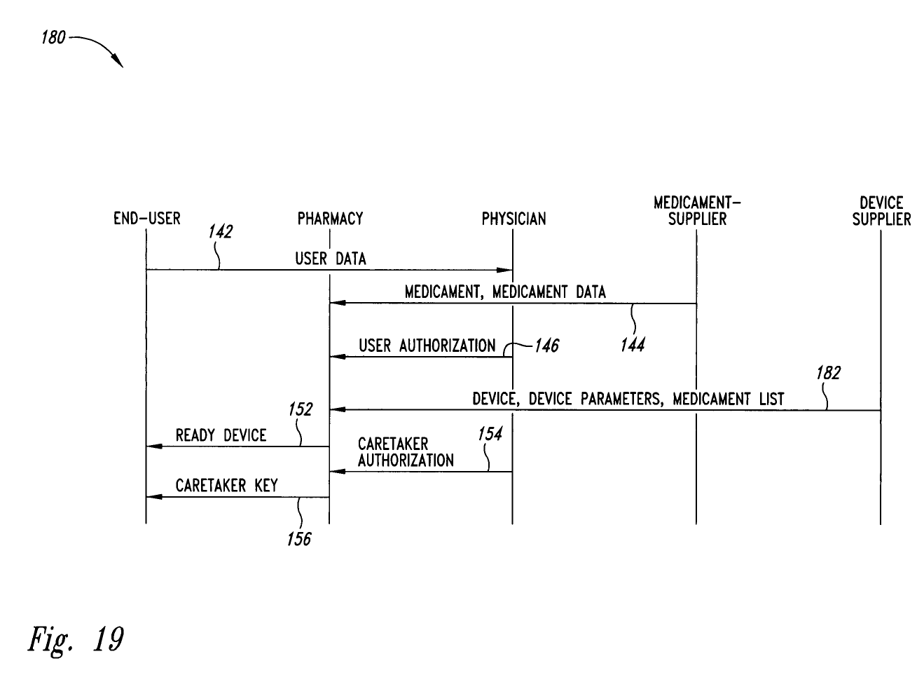

A third scenario 180 is shown in FIG. 19 in which the device parameters are sent from the device supplier to the pharmacy along with the device and the medicament list (transmission 182). The device parameters could be input into the device through the data input 112 by the device supplier to be stored in the data storage 106 for subsequent use by the CPU 104 to program the system 100 once a medicament is chosen. Alternatively, the system 100 could be designated as a single medicament device so that the system is programmed with parameter data in the data storage 106 already used to adjust the medicament output 102 or the medicament output 102 could be otherwise adjusted at the device supplier for a particular medicament.

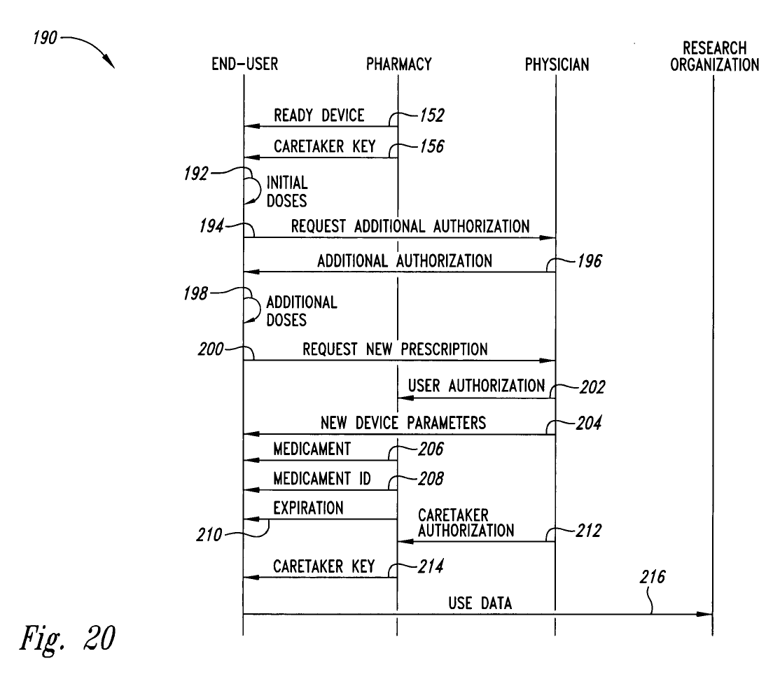

A fourth scenario 190 is shown in FIG. 20 that could be implemented in conjunction with the first scenario 140, the second scenario 160, and/or the third scenario 180 or with other scenarios. After receiving, the ready device (transmission 152) and the caretaker key (transmission 156) if appropriate, the end-user receives an initial number of doses (step 192) that is less than the total number of doses for the associated prescription. Once the initial number of doses is administered, the end-user (or the caretaker if appropriate) sends a request for additional authorization to the physician so that the remaining doses of the prescription can be administered to the end-user (transmission 194). The physician then sends an authorization to administer the additional doses of the prescription to the end-user (transmission 196). The end-user then receives administration of the additional doses of the prescription (step 198).

To manage dose authorization at the sub-prescription level, the system 100 tracks dose usage and locks up use after a certain number of doses are administered. This could, for example, be a series of individually authorized doses. Alternatively, individual serial numbers could be assigned to each individual dose with the system 100 tracking each serial number that is used and only permitting use of each serial number for one time (administration session). The transmission 194 and the transmission 196 can be performed by various ways depending upon how the data output 110 and the data input 112 are configured.

After the initial prescription described in the fourth scenario 190 is used up, the end-user sends a request to the physician for a new prescription (transmission 200). In response, the physician sends a user authorization to the pharmacy (transmission 202). The physician also can send new device parameters to the end-user (transmission 204) either directly to the end-user or to the end-user through the pharmacy depending in part upon how the data output 110 and the data input 112 are configured. The pharmacy sends the end-user the medicament (transmission 206), the medicament identification (transmission 208), and the medicament expiration data (210) by various ways, including those described above, depending upon how the data output 110 and the data input are configured 112.

If a caretaker is involved, the physician sends caretaker authorization to the pharmacy (transmission 212). In turn, the pharmacy sends a caretaker key to the associated caretaker (transmission 214).

In some implementations, a research entity (e.g. clinical trial site) may be involved in collecting data associated with administration by the system 100 of medicament. As shown, the end-user sends use data to a research organization (transmission 216), which could include electronic communication through the data output 110 depending upon its configuration.

The various scenarios were presented as representative examples to illustrate some of the principles involved with some of the components and methods of the system 100. Other scenarios are also involved with the depicted implementations and other implementations of the system 100.

1. A smart device for validating medicament use and optimizing medicament delivery, comprising:
a housing having an aerosolization chamber in which a medicament is aerosolizable, and having, or in communication with a holding means suitable for holding a medicament to be aerosolized;
particle generation means in communication with the aerosolization chamber;
an intelligent interface comprising an input sensing or acquisition means suitable to acquire information from a medicament-associated ID tag, and operative with the intelligent interface to provide for validated medicament delivery, and to provide for at least one of optimization of the particle generating means, and setting of a delivery dosage control parameter, based on the information acquired from the medicament-associated ID tag; and
a user adapter in communication with the aerosolization chamber, the adaptor configured to facilitate delivery of a validated medicament to a user.

2. The device of claim 1, further comprising at least one particle dispersion chamber integral to, or in communication with the aerosolization chamber and the user adapter, to provide for optimized delivery of aerosolized particles to a user.

3. The device of claim 1, wherein the user adapter comprises a nasal, oral, or ocular adapter.

4. The device of any one of claims 1 and 2, wherein the intelligent interface comprises:
a processor and at least one storage device connected thereto, the storage device comprising a stored set of validated medicament identifiers;
and at least one stored software program operative with the processor to receive and process the information from the medicament-associated ID tag, to provide for validated medicament use, and to provide for at least one of optimization of the particle generating means, and setting of a delivery dosage control parameter, based on the information acquired from the medicament-associated ID tag.

5. The device of claim 1, wherein acquiring input information from the medicament-associated ID tag comprises use of at least one of RFID tags, smartcards, barcodes, keyboard entry, voice entry, network interface input, modem interface input, and wireless interface input.

6. The device of claim 1, wherein the medicament-associated ID tag comprises at least one selected from the group consisting of RFID tags, smartcards, barcodes, keyboard entry, voice entry, network interface input, modem interface input, wireless interface input, and combinations thereof.

7. The device of claim 4, wherein medicament use validation comprises:
inputting information from a medicament ID tag;
comparing the input medicament ID tag-associated information with a set of validated medicament identifiers stored in the at least one storage device of the smart device; and
validating or not validating delivery of the medicament, based, at least in part, on the comparison.

8. The device of claim 7, wherein validating or not validating delivery comprises recognition of the presence or absence of a correct medicament product ID.

9. The device of claim 7, wherein validating or not validating delivery comprises recognition of the presence or absence of a non-expired expiration date of the medicament.

10. The device of claim 7, wherein validating or not validating delivery comprises recognition of the presence or absence of a sufficient period of lapsed time since the last medicament delivery.

11. The device of claim 7, wherein validating or not validating delivery comprises recognition of the presence or absence of an inputted caretaker control key required for delivery of a medicament by the device to a user requiring caretaker authorization or assistance to receive the medicament.

12. The device of claim 4, wherein optimization of the particle generating means based on the information acquired from the medicament-associated ID tag comprises adjusting operational parameters selected from the group consisting of: run time; atomization or nebulization rate; generated particle size; linear velocity of the particle; compressor pump output or pulsation pattern; compressor pump speed; compression fluid (e.g., air) pressure; vorticity velocity vectors or vortical flow characteristics; turbulent flow characteristics; dispersion chamber switching or activation/deactivation; variation in relative configuration/orientation and/or distance between a compressed fluid orifice and a liquid feed orifice of a Venturi-type nebulizer or atomizer particle generation means; and combinations thereof.

13. The device of claim 4, wherein the at least one stored software program operative with the processor to receive and process the information from the medicament-associated ID tag, additionally provides for a caretaker control feature, wherein input of a caretaker control key is required for delivery of medicament by the device to a user requiring caretaker authorization or assistance to receive the medicament.

14. The device of claim 4, wherein the at least one stored software program operative with the processor to receive and process the information from the medicament-associated ID tag, additionally provides for storage of a set of historical operational information data in the at least one storage device and further provides for data communication or transmission of the stored historical usage data to PC or PDA devices, smart cards, removable data cartridges, or to one or more authorized or responsible recipients to monitor or manage medicament prescription or administration.

15. A method for validating medicament use and optimizing medicament delivery, using a particle generation and delivery device, comprising:
providing user data by an end-user to a prescribing entity or physician to provide for a prescription for a medicament;
providing the prescription of the prescribing entity or the physician to a prescription drug supplier or pharmacy, the prescription comprising user authorization for use of the prescription medicament by the end-user;
providing, by a medicament supplier, the medicament and associated medicament data to the prescription drug supplier or pharmacy;
providing, by a device supplier, a particle generation and delivery device along with a list of medicaments validated for delivery by the device;
providing device parameters suitable to optimize particle generation and delivery of the prescription medicament by the particle generation and delivery device;
inputting the medicament data and device parameters, and adjusting the particle generation and delivery device according to the device parameters;
recognizing, by the device, the prescription medicament as a validated medicament; and
delivering, using the adjusted particle generation and delivery device, of the validated medicament to the end-user.

16. The method of claim 15, wherein the particle generation and delivery device is adjusted according to the device parameters by the prescription drug supplier or pharmacy, and thereafter provided by the prescription drug supplier or pharmacy to the end-user along with the prescription medicament.

17. The method of claim 16, wherein the device parameters are provided to the prescription drug supplier or pharmacy by the device supplier, by the medicament supplier, or by the prescribing entity or the physician.

18. The method of claim 15, wherein the prescription further comprises a caretaker authorization code or key that must be input into the device to allow for delivery of the validated medicament.

19. The method of claim 15, wherein the medicament data is printed and/or electronic, and comprises at lest one of medicament identification, expiration, pedigree or e-pedigree, and user instructions.

20. The method of claim 15, wherein delivering, using the adjusted particle generation and delivery device, of the validated medicament to the end-user comprises:
delivery of an initially authorized sub-prescription number of doses that is less than the total number of doses for the associated prescription;
requesting, by the end-user, authorization for additional doses from prescribing entity or the physician;
obtaining said authorization; and
delivering of said additional doses to the end-user.

21. The method of claim 15, wherein at least one of:
providing user data by an end-user to a prescribing entity or physician to provide for a prescription for a medicament;
providing the prescription of the prescribing entity or the physician to a prescription drug supplier or pharmacy, the prescription comprising user authorization for use of the prescription medicament by the end-user;
providing, by a medicament supplier, the associated medicament data to the prescription drug supplier or pharmacy;
providing, by a device supplier, a list of medicaments validated for delivery by the device;
providing device parameters suitable to optimize particle generation and delivery of the prescription medicament by the particle generation and delivery device;
inputting the medicament data and device parameters, and adjusting the particle generation and delivery device according to the device parameters; and
recognizing, by the device, the prescription medicament as a validated medicament,
comprises transmission of data over a local area network (LAN), wide area network (WAN), or wireless network.

22. A computer implemented method for validating medicament use by, and optimizing medicament delivery to an end-user, comprising:
configuring, in one or a plurality of electronic databases stored in a storage device of a computerized particle generation and delivery device, a set of medicament validation data for medicaments authorized for delivery by the device, and a set of device parameters suitable to optimize particle generation and delivery of a validated medicament by the particle generation and delivery device;
inputting medicament associated information from a medicament ID tag into the device;
validating, using a software program stored on the storage device that is operative with a processor of the computer to receive and process the medicament associated information to provide a deliverable validated medicament, and is operative to provide for optimizing the particle generation and delivery device according to the device parameters; and
delivering, using the optimized device, the validated medicament to an end-user.

23. The method of claim 22, wherein the set of device parameters suitable to optimize particle generation and delivery of a validated medicament by the particle generation and delivery device is input in to the device along with the medicament associated information.

24. The method of claim 23, wherein the medicament associated information and the device parameters are both part of the medicament ID tag.

25. The method of claim 22, wherein the medicament is a prescription medicament, and the medicament ID tag is attached, imbedded, integral to, or otherwise associated with the prescription medicament to provide for validated medicament use and optimal delivery thereof.

26. The method of claim 25, wherein the particle generation and delivery device is provided to the end-user along with the prescription medicament.
